(19) United States
(12) Patent Application Publication
(10) Pub. No.: US 2026/0086175 A1
(43) Pub. Date: Mar. 26, 2026

(54) SCREEN-PRINTED AND THERMOFORMED ANTENNA FOR SINGLE USE MONITORING DEVICES

(71) Applicant: GE Precision Healthcare LLC, Waukesha, WI (US)

(72) Inventors: Robert Filip Arnold Santala, Uusimaa (FI); Ville Petteri Vartiovaara, Uusimaa (FI)

(21) Appl. No.: 18/898,428

(22) Filed: Sep. 26, 2024

(51) Int. Cl. G01R 33/34; A61B 5/055; G01R 33/341

(52) U.S. Cl. CPC G01R 33/34007; G01R 33/341; A61B 5/055

(57) ABSTRACT

A method for manufacturing a wireless medical device is provided, where the device includes a screen-printed and thermoformed antenna for transmitting physiological, diagnostic, and/or imaging signal information acquired from a patient, such as vital signs from a patient monitor, or receiving a signal of proton realignment within a measurement coil of an MRI machine. The antenna is generated by a conductive trace screen-printed on a substrate using a conductive ink. The substrate is then heated and stretched over a mold, after which a vacuum may be generated underneath the heated substrate. The thermoformed substrate may be configured to follow a contour of a surface of a body of the patient, or to form an enclosure in which integrated circuitry may be positioned, thereby reducing a number of parts of the device. By manufacturing the antennas in this manner, customized antenna structures may be manufactured at low cost, in large quantities.

### TECHNICAL FIELD

Embodiments of the subject matter disclosed herein relate to antennas for wireless monitoring devices and systems.

### BACKGROUND

Monitoring physiological parameters of a patient is an important part of patient care, and physicians often desire to continuously monitor multiple physiological parameters of their patients. For example, well-known parameters of patient health include blood pressure, oxygen saturation (SpO2), and features of the electrocardiogram (ECG). Patient monitoring often involves the use of several sensing devices to perform multiple physiological monitoring modalities, such as a pulse oximeter, a blood pressure monitor, a heart monitor, a temperature monitor, etc. Many patient monitoring devices offer multi-modality patient monitoring, where multiple different sensing devices for sensing different physiological parameters can be connected to a single patient monitor that is configured to collect, process, and/or display physiological information describing the patient's health condition. As a result, a number of sensing devices used to collect and transmit patient data within a hospital environment may be large.

The patient monitor may be connected to wireless network accessible in the hospital environment such that the multiple different sensing devices may communicate with the patient monitor wirelessly. Additionally, a caregiver may monitor physiological patient data of the patient remotely by viewing data of the patient monitor via a remote viewing application (e.g., on a smart phone) as the patient or the caregiver move around a hospital environment. Transmitting physiological patient data from a sensor placed on a patient to the patient monitor may rely on a radio communication antenna of the sensor, which may be coupled to the sensor for transmission of the data. As a result, the large number of sensing devices in use in a typical hospital environment demand an equally large number of antennas. The antennas and the sensing devices themselves may be single use devices that are disposed of after data collection has ended.

Because each antenna is made of a conductive material crefully shaped to resonate on a specific frequency range, a cost of manufacturing the large number of antennas may be high. Additionally, antennas of different sizes, shapes, or configurations may be used for different types of sensors, data, or environments. The complexity entailed by supporting the different sizes, shapes, or configurations may further increase the antennas'manufacturing cost.

### SUMMARY

The current disclosure at least partially addresses one or more of the above identified issues by a method for manufacturing a medical device, such as a diagnostic or monitoring device, the method comprising printing an electrical path on a substrate material using a conductive ink; thermoforming a section of the substrate material including the electrical path using a mold, to create a hollow, non-flat shape; and electrically connecting the electrical path to integrated circuitry of the medical device; wherein the electrical path included on the thermoformed section of the substrate material comprises an antenna relied on by the integrated circuitry to transmit or receive physiological, diagnostic, and/or imaging signal information. In one example, the medical device is a wireless patient monitoring device including a sensor that acquires physiological patient data, and the thermoformed section is used to enclose and protect the integrated circuitry. In another example, the medical device is a measurement coil of a magnetic resonance imaging (MRI) apparatus, which is thermoformed to follow a contour of a surface of a body of a subject of the MRI apparatus.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 schematically illustrates an exemplary patient monitoring environment;

FIG. 2 is a schematic block diagram of an exemplary wireless patient monitoring system including an antenna;

FIG. 3 is a flowchart illustrating an exemplary high-level method for manufacturing a printed, thermoformed antenna of a monitoring device;

FIG. 4 is a flowchart illustrating an exemplary method for printing an antenna on a substrate material;

FIG. 5 is a flowchart illustrating an exemplary method for thermoforming an antenna into an enclosure of a monitoring device;

FIG. 6 is a perspective view of an exemplary thermoformed antenna;

FIG. 7 is a perspective view of a substrate material of an exemplary patient monitoring device including a printed antenna;

FIG. 8 is a perspective view of an exemplary manufactured patient monitoring device including a thermoformed, printed antenna;

FIG. 9 is a schematic diagram of an exemplary magnetic resonance imaging (MRI) system; and

FIG. 10 is an image of an exemplary thermoformed RF measurement coil used in an MRI apparatus.

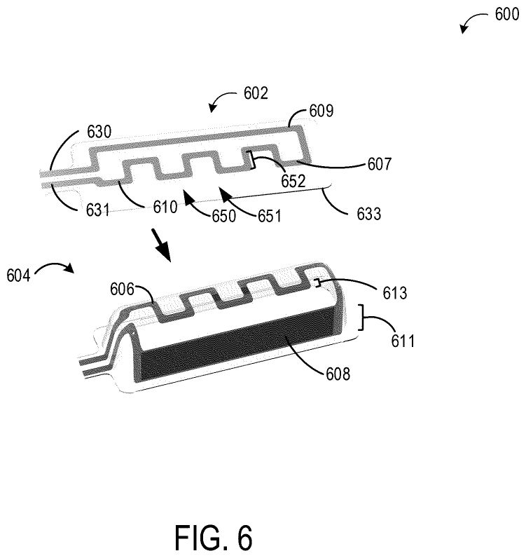

FIGS. 6-8 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. FIGS. 6-8 are shown to scale, although other relative dimensions may be used, if desired.

### DETAILED DESCRIPTION

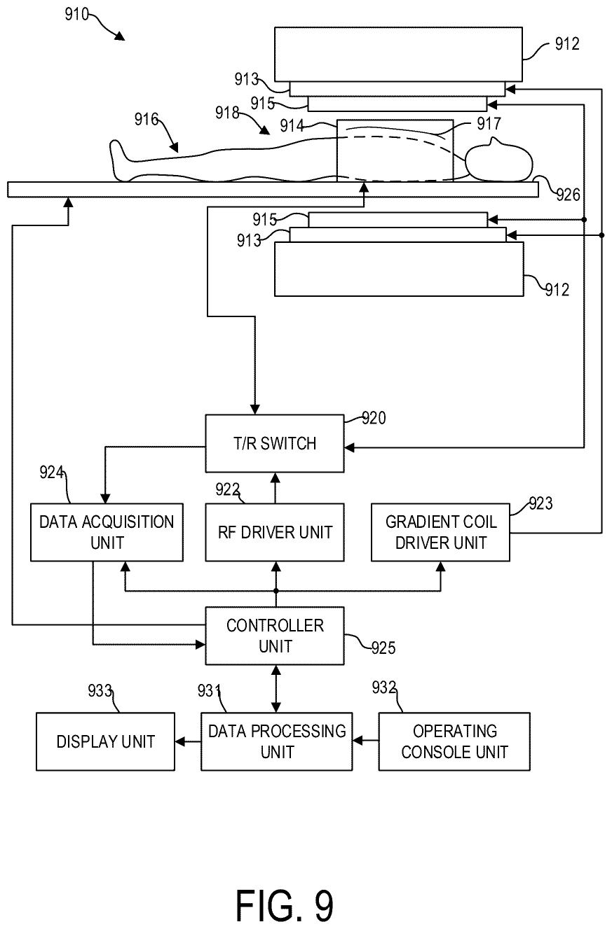

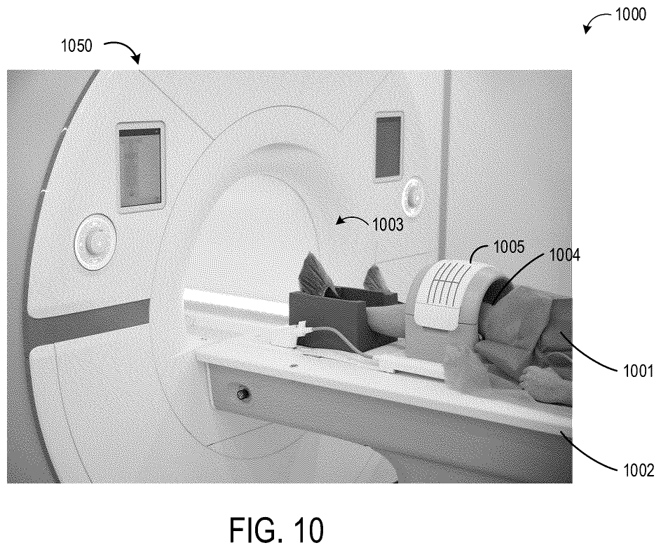

The following description relates to the wireless transmission of data from devices worn by patients, such as sensors of a wireless patient monitoring system, which rely on antennas integrated into the devices. It should be appreciated that while the systems and methods proposed herein are generally described with respect to a patient monitoring system, in other examples, the systems and methods may also be used with different types of medical or other devices used to transmit or receive wireless data, such as, for example, a body coil of a magnetic resonance imaging (MRI) system, as described in relation to FIGS. 9 and 10.

Patient monitoring may include a number of different physiological monitoring devices, sensors, etc. capable of monitoring cardiac, respiratory, neurologic, hemodynamic, pulse oximetry, etc. parameters such as but not limited to electrocardiography (ECG), peripheral capillary oxygen saturation (SpO2), respiration rate, temperature, blood pressures, entropy, blood glucose, and carbon dioxide. Patient monitoring is performed by way of many different forms and approaches with respect to data capture and communication technologies (e.g., hard-wired and wireless networking) and may include monitoring a patient locally (e.g., in-room wired or tethered to a monitor) and/or wirelessly (e.g., in-room, while in transport, ambulating telemetry). In addition to moveable roll stand and room-based semi-fixed or permanently mounted physiological patient data acquisition equipment acquiring one or more parameters, monitoring may be performed with small, portable devices (whether as multiple separate sensors or as an integrated acquisition device) coupled to the patient in order to enable the patient to ambulate (e.g., walk) remotely relative to a designated hospital bed or treatment room while maintaining monitoring of the condition of the patient (e.g., heart rhythm, oxygenation, and other patient vital signs). For example, ambulation of the patient may be desirable for resolution of various medical conditions for which the patient is being treated (e.g., chest pain, syncope, post-surgical). A care provider (e.g., nurse, doctor, or another clinician) may view an output of the monitoring device(s) on the device's user interface, at a remote location such as a patient monitoring central station, via another method such as an Electronic Medical Record (EMR) system, or at a handheld device throughout the duration that the monitoring device(s) is attached or coupled to the patient.

The patient monitor may be connected to a wireless network. The wireless network may further include a patient information database and/or other devices by which medical professionals may access and monitor patient data. Patient monitoring may include receiving physiological patient data from one or more sensors (e.g., sensing components) placed on the patient and displaying the physiological patient data on a patient monitor, and/or uploading the physiological patient data to hospital information systems via a wireless network. Once the physiological patient data has been uploaded to the hospital information systems, the physiological patient data may be transmitted to other devices such as a caregiver device for remote viewing.

The physiological patient data may be transmitted wirelessly from a sensor of the one or more sensors to the other devices via an electrical (radiofrequency) antenna coupled to the sensor. Thus, each sensor used to acquire physiological patient data from each patient may rely on a dedicated electrical antenna. The sensors and/or the antennas may be single-use devices, which are disposed of when the physiological patient data is no longer acquired. Because a number of patients being wirelessly monitored in a health care system may be large, the wireless monitoring may rely on a large number of such disposable devices. However, manufacturing metal electrical antennas at high volume may be costly.

To reduce a cost of manufacturing a high volume of electrical antennas, a method is disclosed herein for manufacturing a low-cost, high-volume electrical antenna by screen-printing a conductive ink on a flat substrate material (e.g., a film). The screen-printed substrate material may then be thermoformed into a hollow, three-dimensional (3D) shape for use in biomedical monitoring and instrumentation devices. In particular, electronics of the biomedical monitoring and instrumentation devices may be advantageously enclosed and housed within the hollow 3D shape. By manufacturing the antennas in this manner, antenna structures that can be customized for a type of sensor, type of data, or type of system may be manufactured at low cost, in large quantities. The disclosed method may also minimize a demand for additional parts, since the thermoformed substrate can simultaneously act as a device enclosure and/or structural support. The minimization of parts also reduces a weight of the antennas, and a size of an end product including a sensor may be decreased. The screen printed and thermoformed antennas may be used with patient monitoring sensors such as pulse oximetry, body temperature, electrocardiographic and respiratory rate sensors, and/or other types of sensors, and/or other types of wireless devices.

The thermoforming allows for creating various antenna geometries, including, but not limited to, dipole, monopole and loop antennas. An electrical two-dimensional path of the antenna may first be screen printed using a conductive ink, such as a silver ink, on a thermoformable plastic sheet, such as polycarbonate, polystyrene or polyethylene terephthalate. After printing, the ink may be partially or totally cured prior to the thermoforming phase, or the ink may be cured by the heat of the thermoforming process. During the thermoforming phase, the sheet may be heated and stretched over a mold, after which a vacuum may be generated at a first side of (e.g., underneath) the heated sheet. An over-pressure may be applied on a second, opposite side (e.g., a top side) of the sheet to further increase a precision and robustness of the thermoforming. After the thermoformed sheet is cooled and removed from the mold, the thermoformed sheet may be bonded to a second plastic sheet to form enclosures in which a sensor or integrated circuitry, such as a printed circuit board assembly, may be placed, or may be bonded to the integrated circuitry itself.

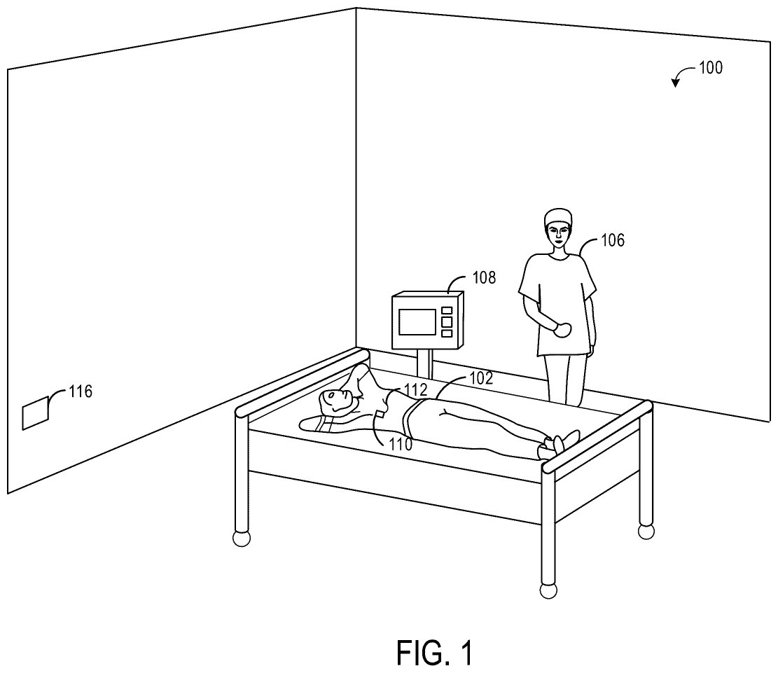

Referring now to the figures, FIG. 1 shows an example patient monitoring environment 100, which in the depicted example is a patient room in a hospital or other medical facility. Patient monitoring environment 100 includes a patient 102 being monitored and attended to by a clinician 106. Clinician 106 may be a nurse, physician, medical technologist, or another suitable medical professional. Patient monitoring environment 100 further includes a patient monitor 108 communicably coupled to a patient monitoring device 110. Patient monitoring device 110 may monitor one parameter or more than one parameter. Due to differing patient conditions and varying patient monitoring demands, one or more patient monitoring devices 110 may be used to support the monitoring demands of the patient and to support monitoring under various conditions (e.g., in room, in transport, patient ambulation, etc.). In some examples, the one or more patient monitors 108 may be included or mounted in a floor, table top, or roll stand module that includes one or more leads or other components coupled to the patient or in wireless communication to a device connected to the patient, in order to monitor one or more parameters of the patient (such as ECG, respiration, blood pressure, carbon dioxide levels, etc.), where the one or more patient monitors 108 may be configured to remain in the patient room. In other examples, the one or more patient monitors 108 may be portable handheld devices that can be carried around a hospital environment by a patient or clinician, which may connect wirelessly to one or more patient monitoring devices 110.

Patient monitoring device 110 may include one or more telemetry devices (with different sensor capabilities) housed in a common module (as shown) or housed in two or more separate modules. Patient monitoring device 110 may be positioned on a body of the patient. Patient monitoring device 110 includes one or more sensors connected to the patient 102 via one or more leads or other components, in order to monitor one or more parameters of the patient (such as ECG, respiration, blood oxygen level, etc.).

The patient monitoring data collected by patient monitoring device 110 may be sent wirelessly (e.g., WiFi, Bluetooth, MBAN) to one or more associated devices for processing, analysis, storage, display, etc., such as patient monitor 108, a central station, patient monitoring database, and/or different patient monitoring system. Patient monitoring device 110 may include an antenna 112 to facilitate wireless transmission between patient monitoring device 110 and the one or more associated devices. The methods of wireless communication used by patient monitoring device 110 and the receiving systems vary widely based on the technology used. In one example communication approach, to facilitate the transfer of the patient monitoring data collected by patient monitoring device 110, patient monitoring environment 100 and nearby areas (e.g., hallways, closets, open spaces) may include one or more access points 116, which may receive from and send information (e.g., wirelessly) to patient monitoring device 110 (e.g., the patient monitoring data, communication status). In some examples, patient monitor 108 may include an access point 116. The access point 116 sends the received information to a processing server, a central station, a telemetry monitoring system, and/or another suitable device. If patient 102 leaves the patient room and moves throughout the medical facility, patient monitoring data collected by patient monitoring device 110 may be sent to other access points located throughout the medical facility. Patient monitoring data collected by patient monitoring device 110 may likewise be sent to a processing and analysis server, the central station, the telemetry monitoring system, and/or another suitable device, via wireless communication with access point 116 or another access point, or via a wired connection. It is understood, this example using a transceiver and access point for data communications is one of many different technologies suitable for sharing acquired patient monitoring data with the associated data processing, analysis, storage, and information viewing system components and infrastructure.

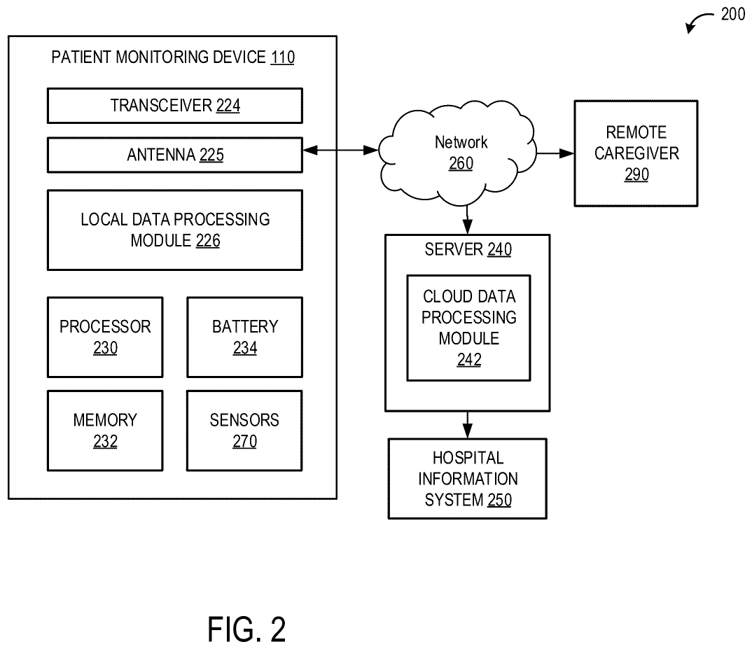

Referring now to FIG. 2, a patient monitoring system 200 is shown that includes patient monitoring device 110 of FIG. 1, which may be connected to a server 240 via a wireless network 260. Patient monitoring device 110 may send data (e.g., including physiological patient data) to server 240 over network 260, and receive data transmitted from server 240 over the network 260. In various embodiments, patient monitoring system 200 may be established within a hospital environment or healthcare facility, such as patient monitoring environment 100 of FIG. 1. In some embodiments, a remote caregiver 290 may view the patient data on patient monitoring device 110 remotely in an office, research module, laboratory, or while moving around the hospital facility, also via network 260.

In some embodiments, patient monitoring device 110 may include one or more sensing components 270, which may be housed within a same enclosure, which is positioned on a patient at a location where the patient is being monitored, as described above in reference to FIG. 1. For example, patient monitoring device 110 may be located within the healthcare facility, and positioned such that the one or more sensing components 270 are in contact with a skin of the patient at a bed of the hospital facility. The patient may wear various patient monitoring devices 110, each measuring different vital signs and each including their own microcontroller, battery, antenna etc.

The one or more sensing components 270 may be specially designed devices for sensing a certain type or types of patient data via placement on a patient's body, and communicating the patient data to patient monitoring device 110. The one or more sensing components 270 may further include a plurality of sensors of different types or the same type. The one or more sensing components 270 may be used to obtain physiological patient data from a patient. For example, the one or more sensing components 270 may include, but are not limited to, a 3-lead ECG sensor, a pulse oximetry sensor, a blood pressure sensor, a digital stethoscope, a respiratory sensor, a temperature sensor, and the like. The one or more sensing components 270 may include a combination of one or more different kinds of sensor.

The physiological patient data is alternatively referred to herein as patient data. The patient data may include, for example, vital signs of the patient, such as a blood pressure or a pulse, and/or any other type of data that may be acquired from a sensor capable of acquiring patient physiological patient data in real-time. Thus, the data sensed by the one or more sensing components 270 correlates to the type of sensors in one or more sensing components 270 and may include ECG data, PPG data, blood pressure data, SpO2 data, respiratory rate, otoscope data, temperature data, and the like. It will be appreciated that the types of sensing components listed above are mentioned for illustrative purposes, and the one or more sensing components 270 may additionally include other types of sensors for obtaining physiological signal information of a patient without departing from the scope of this disclosure.

The patient data obtained from the one or more sensing components 270 may be acquired concurrently or selectively from one or more selected subsets of sensing components of the one or more sensing components 270. In some embodiments, different types of patient data may be acquired concurrently or selectively independent of each other. For example, patient monitoring device 110 may acquire auscultation data (e.g., via a digital stethoscope) but no ECG data, or ECG data but no auscultation data. In other words, while audio and ECG sensors may both be present, auscultation or ECG data may be selectively obtained.

Network 260 may include in a non-limiting manner, a wide area network (WAN); a local area network (LAN); the Internet; a wired or wireless (e.g. optical, Bluetooth, Bluetooth Low Energy (BLE), radio frequency (RF)) network; a cloud-based computer infrastructure of computers, routers, servers, gateways, etc.; or any combination thereof associated therewith that allows patient monitoring device 110 to communicate with other components of patient monitoring system 200. In some examples, patient monitoring device 110 may communicate with the other components via patient monitor 108 of FIG. 1. Network 260 may be or include a public network, or a private network associated with a portion of a care facility, for example a surgery module or department of a hospital, or may be more broadly located across medical devices of an entire hospital or hospital system.

In some embodiments, patient monitoring device 110 and the one or more sensing components 270 may be communicatively coupled via a wireless personal area network (PAN) technology such as a Medical Body Area Network (MBAN). In other embodiments, any PAN technology may be used, such as induction wireless, infrared wireless, ultra wideband (UWB), Bluetooth®, or any other similar technology for wireless communication between co-located devices. For example, the one or more sensing components 270 may communicate with patient monitoring device 110 via an MBAN network of network 260, and patient monitoring device 110 may communicate with other elements of patient monitoring system 200 via a WiFi network.

Patient monitoring device 110 may include a transceiver 224, an antenna 225 (e.g., antenna 112), a local data processing module 226, a processor 230, a memory 232, and a battery 234. Patient monitoring device 110 may be adapted to receive data over the network 260 via antenna 225 and transceiver 224, such as sensor configuration and/or selection data. In some embodiments, the transceiver 224 may be or may include a WLAN wireless card. In some embodiments, the WLAN card may be an original equipment manufacturer (OEM) card, and may include a storage medium having computer executable code and a processor to execute that code, thus effectuating the operation of the WLAN card. In another embodiment the tranciever may be a OEM radio module or system on chip. Patient monitoring device 110 may use transceiver 224 to connect to the network 260 via antenna 225. For example, transceiver 224 may connect to network 260 via an access point of the network (e.g., access point 116 of FIG. 1) arranged at a location of the hospital environment, for example, in proximity to patients being monitored at a care module. The network may receive wirelessly transmitted information from the access point, and relay the information to one or more connected devices and/or a hospital information system suitable for collecting and managing such information.

Patient monitoring device 110 may include a processor 230. The processor 230 may control the operation of patient monitoring device 110. In some embodiments, a user may interact with local data processing module 226 and adjust, configure, and/or select sensing components 270 via control signals sent to the processor 230 via network 260. The processor 230 may execute instructions stored on a memory 232 to control patient monitoring device 110. As discussed herein, the memory 232 may include any non-transitory computer readable medium in which programming instructions are stored. For the purposes of this disclosure, the term “tangible computer readable medium” is expressly defined to include any type of computer readable storage. The example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g. for extended period time periods, permanently, brief instances, for temporarily buffering, and/or for caching of the information). Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include any other medium which can be used to store the desired electronic format of information and which can be accessed by the processor or processors or at least a portion of a computing device. In various embodiments, the memory 232 may include an SD memory card, an internal and/or external hard disk, USB memory device, or similar modular memory.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Upon being received at patient monitoring device 110 from the one or more sensing components 270, the patient data may be processed by local data processing module 226. Processing the patient data may include, for example, comparing values of the patient data with one or more threshold values stored in the memory 232. For example, patient monitoring device 110 may include one or more lookup tables stored in the memory 232 with the one or more threshold values. During processing of the patient data, a threshold value of the one or more threshold values may be retrieved from the one or more lookup tables and compared to a corresponding value of the patient data. For example, if a blood pressure of the patient exceeds a threshold blood pressure (e.g., retrieved from the one or more lookup tables), a health status alert may be generated. In response to the health status alert being generated, an alarm may be generated, to be displayed to remote caregiver 290, for example.

In some embodiments, the patient data and/or results of any processing of the patient data (e.g., health status information) may be transmitted over the network 260 to one or more hospital information systems 250 of the patient monitoring system 200, via server 240. Server 240 may also serve the patient data and/or results to a wireless device of remote caregiver 290, so that the remote caregiver 290 may view the patient data and/or results. Further, some or all of the processing of the patient data may be carried out at server 240. For example, under some conditions, the patient data received at patient monitoring device 110 from the one or more sensing components 270 may be transmitted to server 240 over the network 260 without being processed at local data processing module 226, and the patient data may be processed at a cloud data processing module 242 of server 240.

As described in greater detail below, antenna 225 may be a printed and thermoformed antenna. Additionally, antenna 225 may be printed on and/or thermoformed into an enclosure in which patient monitoring device 110 is housed. By printing and thermoforming antenna 225 in this manner, a size, weight, and a cost of patient monitoring device 110 may be reduced with respect to alternative patient monitoring devices that rely on antennas formed out of sheet metal or other conventional antennas such as chip antennas or etched circuit board antennas. Because patient monitoring device 110 may be a single use, disposable device that is used on a single patient in a single instance and then discarded, the reduction in the cost of the printed and thermoformed antenna may have a large impact on an overall cost of a hospital's patient monitoring demands.

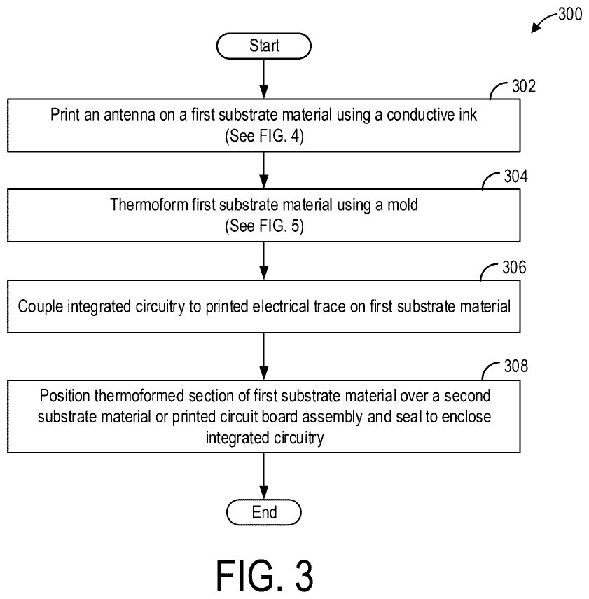

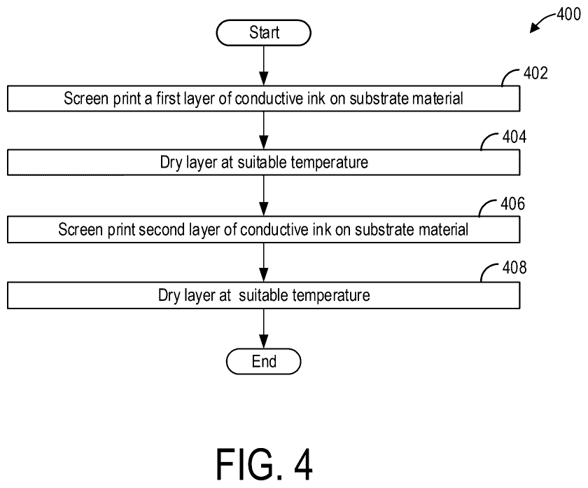

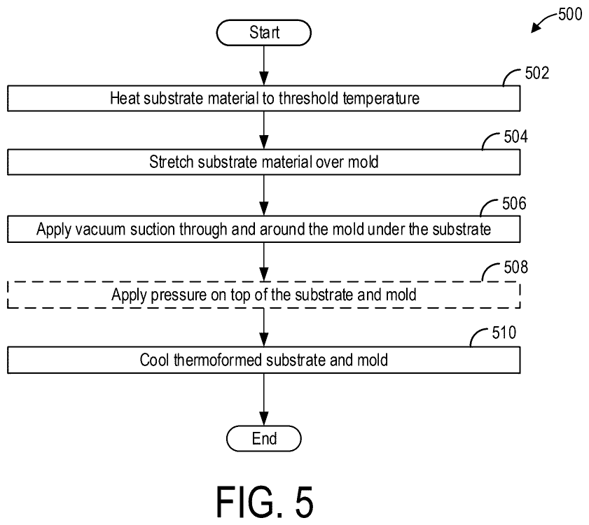

FIG. 3 shows a high-level method 300 for manufacturing a patient monitoring device such as patient monitoring device 110, using printed conductive ink and thermoforming to form an antenna such as antenna 225. Details of the manufacturing process are further elaborated upon in the methods of FIGS. 4 and 5, and perspective views of the patient monitoring device at various stages of the manufacturing process are shown in FIGS. 6-8.

In a first step 302, method 300 includes printing a two-dimensional (2D) design of an antenna on a first substrate material using a conductive ink, thereby forming an electrical path or trace. The conductive ink may also be formed into a plurality of conductive traces through which a sensor of the patient monitoring device may electrically communicate with electrical components of the patient monitoring device such as a transceiver, which may be mounted on an integrated circuitry. Printing the conductive traces on the first substrate material is described in greater detail below in reference to FIG. 4.

In a second step 304, method 300 includes thermoforming the printed first substrate material, or one or more portions of the printed first substrate material using a mold. Thermoforming the first substrate material may include various steps involving stretching the first substrate material and applying vacuum and/or pressure to the first substrate material, which are described in greater detail below in reference to FIG. 5.

After the printed, thermoformed first substrate material has cooled, at a third step 306, method 300 includes coupling integrated circuitry, such as a printed circuit board, to a printed electrical trace of the first substrate material, such that electrical signals generated by the integrated circuitry and/or sensors of the patient monitoring device may be transmitted wirelessly to an external device (e.g., patient monitor 108) via an antenna formed by the printed conductive traces.

At a fourth step 308 of the method, method 300 includes positioning the thermoformed first substrate material over the integrated circuitry and electronic components, and sealing the thermoformed first substrate material to a second substrate material on which the integrated circuitry has been positioned, to enclose the electrical components. Alternatively, the thermoformed first substrate material may be sealed directly to the integrated circuitry. For example, the integrated circuitry may include a printed circuit board, and the thermoformed first substrate material may be sealed to the printed circuit board. The thermoformed first substrate material may be sealed to the integrated circuitry or the second substrate material of the substrate using thermoplastic staking or ultrasonic welding, or using an adhesive or other means of sealing plastic edges of the first and/or second substrates. After the thermoformed first substrate material is sealed around the integrated circuitry, the electrical components may be protected from damage from fluids or contaminants present in the monitoring environment.

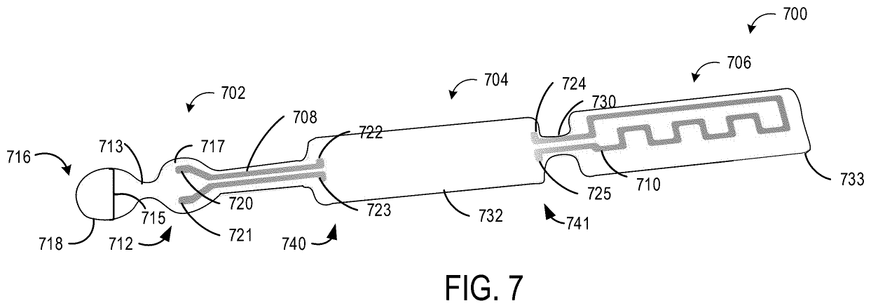

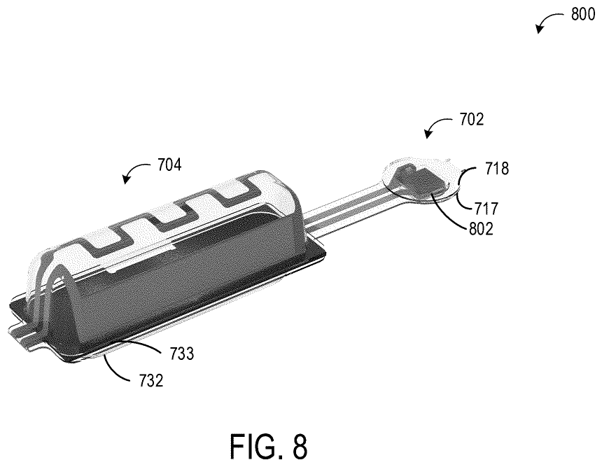

In some examples, the first substrate material may be the same as the second substrate material. That is, the first substrate material may be cut into a shape that allows one or more first portions of the first substrate material to be folded onto one or more second portions of the first substrate material, such that edges of the first portions may be aligned with edges of the second portions to facilitate sealing the first and second portions together. For example, the first substrate material may be divided into various sections, which may then be positioned in face-sharing contact after thermoforming. One or more sensors (e.g., sensing components 270) may be positioned at a first section; a printed circuit board, batteries, etc., may be positioned at a second section; a third section of the substrate material may be thermoformed to create the antenna. After thermoforming, the third section may then be positioned on top of the second section (e.g., folded) to enclose the integrated circuitry. An example of such a design is shown in FIGS. 7-8.

Referring now to FIG. 4, a method 400 is shown for printing a conductive ink on a substrate material to form a printed antenna such as the printed antenna of conductive trace 610 of FIGS. 6, 7, and 8. Method 400 may be performed as part of method 300 of FIG. 3, by a manufacturer of a patient monitoring device such as patient monitoring device 110 of FIGS. 1 and 2.

Method 400 begins at 402, where method 400 includes screen printing a first layer of conductive ink on a substrate material, such as plastic film 700. Certain substrate materials may be more suitable than others. The substrate material may be a plastic film that can endure relatively high temperatures used for ink drying, while still being formable at a higher temperature. For example, an ideal ink may dry at 70° C. with no deformation, but still be thermoformable at 140°. For example, the substrate material may include polyethylene terephthalate glycol (PETG), polystyrene (PS) or high impact polystyrene (HIPS), ethylene-vinyl acetate (EVA) or a polycarbonate (PC) polymer. One advantage of the methods described herein is that the substrate material may be a bio-compatible material, where the bio-compatibility of the material is unaffected by the methods.

The conductive ink used may be selected based on a tolerance of the ink to stretching. In various embodiments, the conductive ink may be a silver-based ink. Screen printing the first layer of conductive ink further includes selecting a first mesh size of a screen printer, which may depend on the selected ink. In some embodiments, an inkjet printer, or a different ink dispensing method, may be used instead of or in addition to screen printing. As a result of the screen printing, one or more printed electrical traces with the conductive ink are positioned on the substrate material, where the printed electrical traces comprise an antenna used by a wireless transmitter to transmit data. The printed electrical traces of the conductive ink form a structure that is bonded to the plastic film, that can be detected or inspected.

At 404, method 400 includes drying the layer. For example, drying the layer may include exposing the ink of the first layer of conductive ink to a suitable temperature to dry the ink without deformation. In one example, the suitable temperature is 70°. When the ink is dried, solvents in the ink may be vaporized. However, the ink may not be cured during the drying process, as fully cured inks may be more resistant to thermoforming.

At 406, method 400 includes screen printing a second layer of conductive ink on the substrate material. The second layer of conductive ink may be printed at the same location(s) on the substrate material as the first layer of conductive ink (e.g., on top of the first layer). The conductive ink used in the second layer may be the same as the conductive ink used in the first layer, or a different conductive ink may be used. Printing a plurality of layers of ink on top of each other may make the conductive traces more tolerant to thermoforming. That is, a first conductive trace comprising a plurality of layers of ink may stretch during the thermoforming process and not break, while a second conductive trace comprising a single layer of ink may break during the thermoforming process. Screen printing the second layer of conductive ink further includes selecting a second mesh size of the screen printer, which may be different from the first mesh size. Using different sized meshes for printing different layers may result in a more robust conductive trace that is more prone to stretching and less prone to breaking.

At 408, method 400 includes drying the ink of the second layer of conductive ink at the suitable temperature, as described above, and method 400 ends. However, it should be appreciated that while for simplicity method 400 includes applying two layers of conductive ink, in other embodiments, a greater number of layers of conductive ink may be printed on the substrate material in accordance with method 400. Additionally or alternatively, additional dielectric layers may be printed on top of the conductive traces of the first and/or second layers of conductive ink, which may lead to better thermoforming results. The dielectric layers can be printed in selected locations to stiffen specific sections during the thermoforming, allowing some areas to stretch more, and some areas to stretch less during the forming phase. The dielectric layers may also be printed to insulate the conductive layers, for example, to avoid short circuiting of battery cells inside the hollow dome space. Method 400 ends.

Referring now to FIG. 5, a method 500 is shown for thermoforming a substrate material on which a conductive trace has been printed to form an enclosure a patient monitoring device, such as patient monitoring device 110 and/or patient monitoring device 800 of FIG. 8. By thermoforming the substrate material, the substrate material may be formed into a hollow shape that may be used to enclose an integrated circuitry and/or other associated electronics of the patient monitoring device, such as integrated circuitry of FIG. 7. Method 500 may be performed as part of method 300 described above in reference to FIG. 3. It should be appreciated that in some embodiments, one or more steps of method 500 may be omitted, or performed in a different order.

Method 500 begins at 502, where method 500 includes heating the substrate material to a threshold temperature. The threshold temperature may be higher than a material glass transition temperature, and a temperature at which the substrate material may be stretched without rupturing, using a mold. For example, the threshold temperature may be 140°. The heating of the substrate material may be performed within a threshold duration, such as five seconds. The heating phase of the thermoforming process may be conducted as quickly as possible, to minimize an effect of curing the ink prior to stretching the substrate material, which may make the ink more resistant to stretching.

At 504, method 500 includes stretching the substrate material over a mold. A size of the mold may be selected based on a size of the integrated circuitry and/or electrical components enclosed within the patient monitoring device. Additionally, a shape of the mold may be designed to facilitate stretching. The mold may be designed such that an elongation of the substrate material is reduced or minimized in printed areas of the substrate material, to reduce a possibility of the printed conductive traces breaking. For example, the mold may be selected from a plurality of molds, where each mold of the plurality of molds is designed for a set of conductive traces or pathways. For each antenna design, a corresponding mold may be selected, where the corresponding mold may have differently shaped portions for areas of the substrate material including conductive traces and areas of the substrate material not including the conductive traces. In this way, when the substrate material is stretched over the selected mold, the areas including the conductive traces may be stretched less than the areas not including the conductive traces.

Additionally, the mold may be selected based on a polarity of the mold. For example, a positive mold may be selected over a negative mold, as the positive mold may result in less stretching than the negative mold. Also, depending on the polarity, the printed traces can be selected to either face towards or away from a surface of the mold. The facing will impact whether the conductive traces are inside or outside the dome substrate, but the stretching of the printed traces may also be different.

At 506, method 500 includes applying a vacuum suction through and around the mold under the substrate. The vacuum suction draws the substrate around the mold. At 508, method 500 optionally includes applying a pressure on top of the substrate material and the mold. Applying the pressure may include holding the pressure for a threshold duration, to ensure that the ink is fully cured and the forming is finished. In one example, the threshold duration is 60 seconds.

At 510, method 500 includes cooling the thermoformed substrate material and the mold, and method 500 ends. Thus, the substrate material may be formed into a desired shape (e.g., of the mold) in three stages, which may be performed sequentially. In the first stage, the substrate material is mechanically stretched; in the second stage, the substrate material is formed by the vacuum; and in the third stage, the substrate material is formed by over-pressure forming. By performing the three stages sequentially, a stretching of the area of the printed conductive trace may be minimized. However, in other embodiments, two or more of the three stages may be performed concurrently.

Turning now to FIG. 6, an exemplary antenna diagram 600 shows an antenna 602, which is created by printing a conductive trace 610 on a substrate 633, in accordance with method 300. Antenna 602 is a loop antenna, where conductive trace 610 forms a closed loop starting from a first end 630 and ending at a second end 631, where first end 630 and second end 631 may be connected to integrated circuitry of a sensing device as described above. However, in other embodiments, conductive trace 610 may form a different antenna design, such as a dipole, monopole, F antenna, planar inverted F antenna, or other antenna geometry.

The printed antenna 602 formed by conductive trace 610 may form a meandering loop that includes a plurality of protruding rectangular sections 650, which may be separated by a plurality of recessed sections 651 by a distance 652. In other embodiments, the number of protruding rectangular sections 650 may be different, and/or the distance 652 between the protruding rectangular sections 650 and the recessed sections 651 may be different.

Thermoforming antenna 602 (e.g., in accordance with method 300) may result in a thermoformed antenna 604, where substrate 633 is stretched and molded to form an enclosure for components of a device, such as a patient monitoring device. Portions of conductive trace 610 may be stretched, while other portions of conductive trace 610 may be stretched. In FIG. 6, a first, meandering side 607 of conductive trace 610 forms a printed electrical path 606 at a top of the enclosure of thermoformed antenna 604 that is not stretched. Alternatively, a straight side 609 of conductive trace 610 forms a printed electrical path 608 at a side of the enclosure of thermoformed antenna 604 that is stretched such that a first width (height) 611 of printed electrical path 608 is greater than a second width 613 of printed electrical path 606. Thus, characteristics of a printed electronic path forming an antenna for the enclosed circuitry depend on where conductive trace 610 is positioned on substrate 633.

An advantage of this antenna design is that the shape and positioning of conductive trace 610 can be simply variated by modifying the printing screen and custom designed to be ideal for internal circuitry of an associated device. The associated device could also include batteries under the dome to power the device and they would impact the design of the antenna. A fairly complex antenna design can by these means be created using existing simple machinery, namely screen printing and thermoforming equipment.

FIGS. 7 and 8 show an exemplary configuration of a patient monitoring device enclosed in a thermoformed antenna such as antenna 604, before and after thermoforming.

Referring to FIG. 7, an exemplary plastic film 700 comprising a 2D sheet of substrate material is shown, which has a suitable shape for incorporating one or more sensors and an integrated circuitry during assembly. While plastic film 700 is depicted as having been cut to form the suitable shape, in practice plastic film 700 may be cut after thermoforming has been performed. The suitable shape has three sections: a first section 702 in which one or more sensors may be positioned; a second section 704 in which an integrated circuitry may be positioned; and a third section 706 including a printed antenna, which may be the same as or similar to antenna 602 of FIG. 6. In other embodiments, the suitable shape may be different, and may include different sections, and/or a different number of sections.

First section 702 of plastic film 700 includes a first portion 712 on which the one or more sensors may be placed, and a second portion 716 which may form a top portion of an enclosure of the one or more sensors. In other examples, sensing elements may sit directly on a printed circuit board assembly, and first section 702 may not be included. In FIG. 7, first portion 712 and second portion 716 have a circular shape of an identical or similar size. In other embodiments, first portion 712 and second portion 716 may have a different shape, such as a rectangular, square, oval, or other shape. A diameter 715, or a size of first portion 712 and second portion 716 may depend on a size of the one or more sensors positioned at section 702. That is diameter 715 (the size) may be larger than a diameter or size of the one or more sensors, such that when the one or more sensors are placed on first portion 712 and enclosed by second portion 716, edges of first portion 712 and second portion 716 may be in face-sharing contact around the one or more sensors, to facilitate sealing the one or more sensors at the edges.

In various embodiments, first portion 712 may be connected to second portion 716 via a connector portion 713, such that second portion 716 may be folded and placed on top of first portion 712 after the one or more sensors have been positioned on first portion 712. Further, in some examples, second portion 716 may be thermoformed as described below in reference to FIG. 5 into a hollow, non-flat or domed shape, which may be positioned on top of first portion 712, with the one or more sensors arranged between first portion 712 and second portion 716 within the hollow domed shape. A first circumferential edge 717 along a perimeter of first portion 712 may be in face sharing contact with a second circumferential edge 718 along a perimeter of second portion 716. First circumferential edge 717 may be coupled to second circumferential edge 718 to seal first section 702 around the one or more sensors, using thermoplastic staking, ultrasonic welding, or a different method.

A first printed electrical path 708 may connect the one or more sensors of first section 702 to an integrated circuitry positioned at or on section 704. The one or more sensors may be electrically connected to a first terminal 720 and a second terminal 721 of first printed electrical path 708 at first section 702. The integrated circuitry may be electrically connected to first printed electrical path 708 at second section 704 via a third terminal 722 and a fourth terminal 723 at a first side 740 of second section 704.

A second printed electrical path 710 may connect to the integrated circuitry at section 704, and may form the printed antenna at third section 706. The integrated circuitry may be electrically connected to the second printed electrical path 710 via a fifth terminal 724 and a sixth terminal 725 at a second side 741 of second section 704.

In accordance with the method of FIG. 5, third section 706 may be thermoformed, after second printed electrical path 710 has been printed, into a hollow shape that may enclose the integrated circuitry in second section 704. That is, third section 706 may be attached to second section 704 via a connector portion 730 of plastic film 700, such that third section 706 may be folded and/or placed on top of second section 704, after third section 706 is thermoformed, with the integrated circuitry sandwiched between second section 704 and third section 706. A first edge 732 along a perimeter of second section 704 may be in face sharing contact with a second edge 733 along a perimeter of third section 706. First edge 732 may be coupled to second edge 733 to seal the integrated circuitry between third section 706 and second section 704, using thermoplastic staking, ultrasonic welding, or a different method.

FIG. 8 shows a perspective view of a fully formed patient monitoring device 800 created using plastic film 700, after manufacturing. The thermoformed, printed antenna at third section 706 of FIG. 7 forms an enclosure that has been positioned (e.g., folded) on top of integrated circuitry (not shown in FIG. 8), such that first edge 732 of second section 704 and second edge 733 of third section 706 are sealed in face sharing contact, thereby protecting the integrated circuitry and associated electronics from contact with external elements. That is, the integrated circuitry has been positioned at second section 704 of the substrate, in electronic communication with third terminal 722 and fourth terminal 723 at first side 740 and with fifth terminal 724 and sixth terminal 725 at second side 741. The associated electronics may include battery cells that power the device, which may be protected by the sealed shape.

Similarly, a sensing electrical component (e.g., a photodetector) 802 has been positioned at first portion 712 of first section 702 and attached to first terminal 720 and second terminal 721. Second portion 716 of first section 702 forms an enclosure that has been positioned (e.g., folded) on top of first portion 712, such that first circumferential edge 717 of first portion 712 and second circumferential edge 718 of second portion 716 are sealed in face sharing contact, thereby protecting photodetector 802 and associated electronics from contact with external elements. Photodetector 802 may be used as part of a pulse oximetry measurement, for example.

Further, in some examples, electrical components and chips may be assembled directly on a screen printed substrate, further reducing a cost and increasing an efficiency of manufacturing. Printed electronics is a well-known technology that could be combined with thermoforming to further save cost and reduce complexity by eliminating a reliance on a separate circuit board.

As described above, the methods described herein may be used to manufacture various types of patient monitoring and/or sensing devices, in particular, where lightweight antennas are desired to be customized to a specific task, technology or patient and the cost of such customization may be high for alternative types of metal antennas. As another example, methods 3, 4, and 5 may be used to manufacture customizable MRI measurement coils that may be used to measure the realigning of protons after gradient stimulation during an MR imaging scan from within an MRI machine. In other words, in an MR application, the antenna functions not as a data transmission antenna as with a patient monitoring device, but rather as an imaging antenna. Due to the nature of MRI technologies, to get sharp and detailed MR images, it may be desired that a measurement coil be tightly fitted around a portion of the patient being scanned. In such situations, a printed and thermoformed antenna may be created that meets the demands for such a close fit more efficiently and inexpensively than alternative options.

Magnetic resonance imaging (MRI) is a medical imaging modality that can create images of the inside of a human body without using x-rays or other ionizing radiation. MRI systems include a superconducting magnet to create a strong, uniform, static magnetic field B₀. When a human body, or part of a human body, is placed in the magnetic field B₀, the nuclear spins associated with the hydrogen nuclei in tissue water become polarized, wherein the magnetic moments associated with these spins become preferentially aligned along the direction of the magnetic field B₀, resulting in a small net tissue magnetization along that axis. MRI systems also include gradient coils that produce smaller amplitude, spatially-varying magnetic fields with orthogonal axes to spatially encode the magnetic resonance (MR) signal by creating a signature resonance frequency at each location in the body. The hydrogen nuclei are excited by a radio frequency signal at or near the resonance frequency of the hydrogen nuclei, which add energy to the nuclear spin system. As the nuclear spins relax back to their rest energy state, they release the absorbed energy in the form of an RF signal. This RF signal (or MR signal) is detected by one or more measurement coils placed tightly around the body (also referred to herein as RF coil units) and transmitted to a computer for processing, where the RF signal is transformed into the image using reconstruction algorithms. The RF signal may be received using a thermoformed antenna, as described below in reference to FIG. 10.

FIG. 9 illustrates a magnetic resonance imaging (MRI) apparatus 910 that includes a magnetostatic field magnet unit 912, a gradient coil unit 913, an RF coil unit 914, an RF body coil unit 915, a transmit/receive (T/R) switch 920, an RF driver unit 922, a gradient coil driver unit 923, a data acquisition unit 924, a controller unit 925, a patient table or bed 926, a data processing unit 931, an operating console unit 932, and a display unit 933. In some embodiments, the RF coil unit 914 is a surface coil, which is a local coil typically placed proximate to the anatomy of interest of a subject 916. Herein, the RF body coil unit 915 is a transmit coil that transmits RF signals, and the local surface RF coil unit 914 receives the MR signals. As such, the transmit body coil (e.g., RF body coil unit 915) and the surface receive coil (e.g., RF coil unit 914) are separate but electromagnetically coupled components. The MRI apparatus 910 transmits electromagnetic pulse signals to the subject 916 placed in an imaging space 918 with a static magnetic field formed to perform a scan for obtaining magnetic resonance signals from the subject 916. One or more images of the subject 916 can be reconstructed based on the magnetic resonance signals thus obtained by the scan.

The magnetostatic field magnet unit 912 includes, for example, an annular superconducting magnet, which is mounted within a toroidal vacuum vessel. The magnet defines a cylindrical space surrounding the subject 916 and generates a constant primary magnetostatic field B₀.

The MRI apparatus 910 also includes a gradient coil unit 913 that forms a gradient magnetic field in the imaging space 918 so as to provide the magnetic resonance signals received by the RF coil arrays with three-dimensional positional information. The gradient coil unit 913 includes three gradient coil systems, each of which generates a gradient magnetic field along one of three spatial axes perpendicular to each other, and generates a gradient field in each of a frequency encoding direction, a phase encoding direction, and a slice selection direction in accordance with the imaging condition. More specifically, the gradient coil unit 913 applies a gradient field in the slice selection direction (or scan direction) of the subject 916, to select the slice; and the RF body coil unit 915 or the local RF coil arrays may transmit an RF pulse to a selected slice of the subject 916. The gradient coil unit 913 also applies a gradient field in the phase encoding direction of the subject 916 to phase encode the magnetic resonance signals from the slice excited by the RF pulse. The gradient coil unit 913 then applies a gradient field in the frequency encoding direction of the subject 916 to frequency encode the magnetic resonance signals from the slice excited by the RF pulse.

The RF coil unit 914 is disposed, for example, to enclose the region to be imaged of the subject 916. In some examples, the RF coil unit 914 may be referred to as the surface coil or the receive coil. In the static magnetic field space or imaging space 918 where a static magnetic field B₀ is formed by the magnetostatic field magnet unit 912, the RF body coil unit 915 transmits, based on a control signal from the controller unit 925, an RF pulse that is an electromagnet wave to the subject 916 and thereby generates a high-frequency magnetic field B₁. This excites a spin of protons in the slice to be imaged of the subject 916. The RF coil unit 914 receives, as a magnetic resonance signal, the electromagnetic wave generated when the proton spin thus excited in the slice to be imaged of the subject 916 returns into alignment with the initial magnetization vector. In some embodiments, the RF coil unit 914 may transmit the RF pulse and receive the MR signal. In other embodiments, the RF coil unit 914 may be used for receiving the MR signals, but not transmitting the RF pulse.

The RF body coil unit 915 is disposed, for example, to enclose the imaging space 918, and produces RF magnetic field pulses orthogonal to the main magnetic field B₀ produced by the magnetostatic field magnet unit 912 within the imaging space 918 to excite the nuclei. In contrast to the RF coil unit 914, which may be disconnected from the MRI apparatus 910 and replaced with another RF coil unit, the RF body coil unit 915 is fixedly attached and connected to the MRI apparatus 910. Furthermore, whereas local coils such as the RF coil unit 914 may be configured to transmit to or receive signals from a localized region of the subject 916, the RF body coil unit 915 generally has a larger coverage area. For example, the RF body coil unit 915 may be used to transmit or receive signals to the whole body of the subject 916, and the RF coil unit 914 may be configured to transmit or receive signals from a specific anatomy of the subject 916, such as a limb, or an organ. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 914 and/or the RF body coil unit 915 depends on the imaging application.

To transmit to or receive signals from the localized region of the subject 916, RF coil unit 914 may be configured to include an RF antenna 917. The RF antenna may be thermoformed as described herein to more closely follow a contour of a portion of an anatomy of the subject 916. For example, the RF antenna 917 may be thermoformed to wrap around a torso, head, leg, foot, etc. of the patient. By thermoforming the RF antenna 917, a distance between the RF antenna 917 and the body of the patient may be reduced, resulting in sharper and more detailed images. Additionally, a thermoformed RF antenna 917 may be lighter and less expensive to manufacture/assemble than other types of RF antennas currently in use.

The T/R switch 920 can selectively electrically connect the RF body coil unit 915 to the data acquisition unit 924 when operating in receive mode, and to the RF driver unit 922 when operating in transmit mode. Similarly, the T/R switch 920 can selectively electrically connect the RF coil unit 914 to the data acquisition unit 924 when the RF coil unit 914 operates in receive mode, and to the RF driver unit 922 when operating in transmit mode. When the RF coil unit 914 and the RF body coil unit 915 are both used in a single scan, for example if the RF coil unit 914 is configured to receive MR signals and the RF body coil unit 915 is configured to transmit RF signals, then the T/R switch 920 may direct control signals from the RF driver unit 922 to the RF body coil unit 915 while directing received MR signals from the RF coil unit 914 to the data acquisition unit 924. The coils of the RF body coil unit 915 may be configured to operate in a transmit-only mode or a transmit-receive mode. The coils of the local RF coil unit 914 may be configured to operate in a transmit-receive mode or a receive-only mode.

The RF driver unit 922 includes a gate modulator (not shown), an RF power amplifier (not shown), and an RF oscillator (not shown) that are used to drive the RF coils (e.g., RF body coil unit 915) and form a high-frequency magnetic field in the imaging space 918. The RF driver unit 922 modulates, based on a control signal from the controller unit 925 and using the gate modulator, the RF signal received from the RF oscillator into a signal of predetermined timing having a predetermined envelope. The RF signal modulated by the gate modulator is amplified by the RF power amplifier and then output to the RF body coil unit 915.

The gradient coil driver unit 923 drives the gradient coil unit 913 based on a control signal from the controller unit 925 and thereby generates a gradient magnetic field in the imaging space 918. The gradient coil driver unit 923 includes three systems of driver circuits (not shown) corresponding to the three gradient coil systems included in the gradient coil unit 913.

The data acquisition unit 924 includes a pre-amplifier (not shown), a phase detector (not shown), and an analog/digital converter (not shown) used to acquire the magnetic resonance signals received by the RF coil unit 914. In the data acquisition unit 924, the phase detector phase detects, using the output from the RF oscillator of the RF driver unit 922 as a reference signal, the magnetic resonance signals received from the RF coil unit 914 and amplified by the pre-amplifier, and outputs the phase-detected analog magnetic resonance signals to the analog/digital converter for conversion into digital signals. The digital signals thus obtained are output to the data processing unit 931.

The MRI apparatus 910 includes a table 926 for placing the subject 916 thereon. The subject 916 may be moved inside and outside the imaging space 918 by moving the table 926 based on control signals from the controller unit 925.

The controller unit 925 includes a computer and a recording medium on which a program to be executed by the computer is recorded. The program when executed by the computer causes various parts of the apparatus to carry out operations corresponding to pre-determined scanning. The recording medium may comprise, for example, a ROM, flexible disk, hard disk, optical disk, magneto-optical disk, CD-ROM, or non-volatile memory card. The controller unit 925 is connected to the operating console unit 932 and processes the operation signals input to the operating console unit 932 and furthermore controls the table 926, RF driver unit 922, gradient coil driver unit 923, and data acquisition unit 924 by outputting control signals to them. The controller unit 925 also controls, to obtain a desired image, the data processing unit 931 and the display unit 933 based on operation signals received from the operating console unit 932.

The operating console unit 932 includes user input devices such as a touchscreen, keyboard and a mouse. The operating console unit 932 is used by an operator, for example, to input such data as an imaging protocol and to set a region where an imaging sequence is to be executed. The data about the imaging protocol and the imaging sequence execution region are output to the controller unit 925.

The data processing unit 931 includes a computer and a recording medium on which a program to be executed by the computer to perform predetermined data processing is recorded. The data processing unit 931 is connected to the controller unit 925 and performs data processing based on control signals received from the controller unit 925. The data processing unit 931 is also connected to the data acquisition unit 924 and generates spectrum data by applying various image processing operations to the magnetic resonance signals output from the data acquisition unit 924.

The display unit 933 includes a display device and displays an image on the display screen of the display device based on control signals received from the controller unit 925. The display unit 933 displays, for example, an image regarding an input item about which the operator inputs operation data from the operating console unit 932. The display unit 933 also displays a two-dimensional (2D) slice image or three-dimensional (3D) image of the subject 916 generated by the data processing unit 31. FIG. 10 shows an image 1000 of an MRI apparatus 1050 such as MRI apparatus 910 of FIG. 9. In image 1000, a patient 1001 is positioned on a table 1002 in preparation for insertion into a cavity 1003 of MRI apparatus 1050. In image 1000, an RF coil unit 1005 (e.g., RF coil unit 914) is arranged around a leg 1004 of patient 1001. RF coil unit 1005 may be used as an imaging antenna to measure the realigning of protons after gradient stimulation of leg 1004 during an MR imaging scan. In contrast to current RF coil units, a screen-printed, thermoformed RF coil unit 1005 may be lighter and thinner, such that RF coil unit 1005 may be advantageously positioned on or coupled to leg 1004 in a manner that occupies a very small amount of the space available around leg 1004 when patient 1001 is positioned within cavity 1003. As a result, an interference of RF coil unit 1005 with patient 1001 and other components of table 1002 and/or MRI apparatus 1050 may be reduced or minimized with respect to other wired or wireless solutions. Further, by screen printing and thermoforming RF coil unit 1005, a cost of RF coil unit 1005 may be reduced. Reducing the cost of the RF coil unit 1005 may enable the manufacturing of disposable RF coil units 1005 that can be custom-fit to an anatomy of a patient or set of patients. As a result of having a tighter, more customized coil, image quality may be increased. Additionally, an imaging sequence may be shortened, which is valuable for MRI usage since a throughput of patients can be increased. An additional benefit is that due to the reduction in costs, coils of various sizes may be created, which may increase the image quality for patients of different sizes and shapes. Further, a comfort of a patient may be increased by using a simpler and more light weight coil, particularly with respect to head coils.

Thus, systems and methods are described herein for manufacturing optimized, low-cost antenna solutions that enable low-cost, disposable devices to communicate wirelessly with patient monitoring systems and/or other receiving computer systems. For example, the methods have been developed for use with patient monitoring sensors such as pulse oximetry, body temperature, electrocardiographic and respiratory rate sensors, and MRI customized measurement coil units. The methods provide a solution for cost effectively manufacturing customized antenna geometries in high volumes not feasible with conventional antenna designs. By screen printing and thermoforming the antennas, it is possible to create antenna geometries including but not limited to dipole, monopole and loop antennas. The antennas may be incorporated into an enclosure used to house and protect electrical components of the patient monitoring sensors, thereby reducing an amount of parts and material used to manufacture the antennas, in comparison with conventional or alternative antenna designs and materials. The antennas may also be thermoformed to follow a contour of a surface, such as a surface of a patient anatomy in an MR machine, to generate sharper and more detailed MR images of the patient anatomy.

The electrical two-dimensional path of the screen printed antenna may be formed with a conductive ink (such as a silver ink) on a thermoformable plastic sheet (such as polycarbonate, polystyrene or polyethylene terephthalate). After printing, the ink may be cured or partially cured, or left completely uncured before the thermoforming phase, where in the latter cases the heat of the thermoforming process will cure the printed ink. During the thermoforming phase, the sheet may be heated and stretched over a mold, after which a vacuum is pumped underneath the heated sheet and an over-pressure may be applied on the top side of the sheet to further improve the forming accuracy. In this way, patient monitoring demands of a hospital or health care system may be met in a cost-effective manner, and with a greater degree of customization than is feasible using conventional antenna designs. Further, the greater degree of customization may facilitate more accurate measurements of physiological, diagnostic, and/or imaging signal information, leading to more accurate diagnoses and improved patient outcomes. Specifically, patient outcomes may be improved by increasing the coverage of vital signs monitoring within the hospital, while still enabling patient mobility to support recovery. Mobility is enabled by wireless monitoring and body-worn sensors, which benefit from a small size and light weight, where the low cost of thermoforming makes it an accessible technology for a larger portion of the patients. Further, in some examples, the thermoformed devices/antennas described herein may be used in home care, meaning, outside hospital premises. In such cases the antenna could be a cellular, LTE, BLUETOOTH, NFC, or 5G antenna.

The technical effect of screen-printing and thermoforming an antenna on a plastic sheet is that a cost of manufacturing the antenna may be less than a cost of conventional or alternative antenna designs, and a degree of customization of the antenna to a patient, task, or technology may be increased, resulting in more accurate measurements of physiological, diagnostic, and/or imaging signal information. Thermoformed antennas may be lighter and of a smaller size, where a substrate simultaneously functions as an enclosure, whereby materials can be environmentally friendly and recyclable (or at least friendlier than conventional PCB antenna manufacturing). The high-level and simplicity of customization may enable more specialized purpose intended antennas that create better wireless links than a generic antenna. This in turn may enable lower RF power and less power consumption in the device.

The disclosure also provides support for a method for manufacturing a medical device, the method comprising: printing an electrical path on a substrate material using a conductive ink, thermoforming a section of the substrate material including the electrical path using a mold, to create a non-flat shape, and electrically connecting the electrical path to integrated circuitry of the medical device, wherein the electrical path included on the thermoformed section of the substrate material comprises an antenna relied on by the integrated circuitry to transmit or receive physiological, diagnostic, and/or imaging signal information. In a first example of the method, the medical device is a wireless patient monitoring device including a sensor that acquires physiological signal information. In a second example of the method, optionally including the first example, the medical device is a measurement coil of a magnetic resonance imaging (MRI) apparatus, the measurement coil thermoformed to follow a contour of a surface of a body of a subject of the MRI apparatus. In a third example of the method, optionally including one or both of the first and second examples, the method further comprises: positioning the integrated circuitry of the wireless patient monitoring device between the thermoformed section of the substrate material and a non-thermoformed portion of the wireless patient monitoring device, and sealing the thermoformed section and the non-thermoformed portion to enclose the integrated circuitry, wherein the non-thermoformed portion of the wireless patient monitoring device is one of: a non-thermoformed section of the substrate material, a non-thermoformed section of a second substrate material, and a printed circuit board of the integrated circuitry. In a fourth example of the method, optionally including one or more or each of the first through third examples, the thermoformed section and the non-thermoformed portion are sealed using one of an adhesive, thermoplastic staking, and ultrasonic welding. In a fifth example of the method, optionally including one or more or each of the first through fourth examples, printing the electrical path on the substrate material using the conductive ink further comprises: screen-printing a first layer of conductive ink on the substrate material, drying the first layer at a first threshold temperature, screen-printing a second layer of conductive ink on the substrate material on top of the first layer, and drying the second layer at the first threshold temperature. In a sixth example of the method, optionally including one or more or each of the first through fifth examples, the method further comprises: printing one or more additional dielectric layers on top of the conductive ink of the first and/or second layers of conductive ink. In a seventh example of the method, optionally including one or more or each of the first through sixth examples, thermoforming the section of the substrate material including the electrical path using the mold further comprises: heating the substrate material to a second threshold temperature, stretching the substrate material over the mold, in a first thermoforming stage, applying a vacuum suction through and around the mold at a first side of the substrate material, in a second thermoforming stage, and applying a pressure at a second, opposite side of the substrate material, in a third thermoforming stage. In a eighth example of the method, optionally including one or more or each of the first through seventh examples, the first thermoforming stage, the second thermoforming stage, and the third thermoforming stage are performed either concurrently or sequentially. In a ninth example of the method, optionally including one or more or each of the first through eighth examples, the mold is selected from a plurality of molds based on the printed electrical path, the mold having differently shaped portions for areas of the printed electrical path and areas of the substrate material not including the printed electrical path, such that when the substrate material is stretched over the selected mold, the areas including the printed electrical path may be stretched less than the areas not including the printed electrical path. In a tenth example of the method, optionally including one or more or each of the first through ninth examples, the substrate material is one of polyethylene terephthalate glycol (PETG), polystyrene or high impact polystyrene (HIPS), ethylene-vinyl acetate, and a polycarbonate (PC) polymer, and the conductive ink is a silver-based ink.

The disclosure also provides support for a wireless patient monitoring device, comprising: an integrated circuitry including a sensing component and a wireless transmitter, wherein the integrated circuitry is enclosed in a plastic film on which one or more printed electrical traces with a conductive ink are positioned, where the printed electrical traces comprise an antenna used by the wireless transmitter to transmit data acquired by the sensing component to a wireless receiver outside the wireless patient monitoring device. In a first example of the system, the plastic film enclosing the integrated circuitry is thermoformed to create a hollow, domed shape in which the integrated circuitry is enclosed. In a second example of the system, optionally including the first example, the sensing component is enclosed within a second thermoformed portion of the plastic film. In a third example of the system, optionally including one or both of the first and second examples, the wireless patient monitoring device is manufactured in a plurality of stages, including: a first stage in which the one or more printed electrical traces are printed on a substrate material in the conductive ink, a second stage in which a section of the substrate material including the one or more printed electrical traces comprising the antenna is heated and mechanically stretched using a mold, a third stage in which a vacuum is applied through and around the mold at a first side of the substrate material, and a fourth stage in which the integrated circuitry is coupled to, enclosed, and sealed by a thermoformed portion of the substrate material created by the second and third stages. In a fourth example of the system, optionally including one or more or each of the first through third examples, the third stage further comprises applying a pressure at a second, opposite side of the substrate material. In a fifth example of the system, optionally including one or more or each of the first through fourth examples in which the substrate material includes a first foldable portion that may be folded over the sensing component to enclose the sensing component, and a second foldable portion that may be folded over the integrated circuitry to enclose the integrated circuitry. In a sixth example of the system, optionally including one or more or each of the first through fifth examples, the mold is configured such that when the substrate material is stretched over the mold, some portions of the one or more printed electrical traces are stretched more than other portions of the one or more printed electrical traces.

The disclosure also provides support for a wireless radio-frequency (RF) coil unit of a magnetic resonance imaging (MRI) machine, comprising: a substrate material on which one or more printed electrical traces of a conductive ink are positioned, wherein the one or more printed electrical traces comprise an imaging antenna that measures a realigning of protons within a portion of an anatomy of a subject of the MRI machine after gradient stimulation of the portion during an MR imaging scan, the substrate material thermoformed to follow a contour of a surface of the portion of the anatomy. In a first example of the system, the wireless RF coil unit is customized to the subject.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method for manufacturing a medical device, the method comprising:
printing an electrical path on a substrate material using a conductive ink;
thermoforming a section of the substrate material including the electrical path using a mold, to create a non-flat shape; and
electrically connecting the electrical path to integrated circuitry of the medical device;
wherein the electrical path included on the thermoformed section of the substrate material comprises an antenna relied on by the integrated circuitry to transmit or receive physiological, diagnostic, and/or imaging signal information.

2. The method of claim 1, wherein the medical device is a wireless patient monitoring device including a sensor that acquires physiological patient data.

3. The method of claim 1, wherein the medical device is a measurement coil of a magnetic resonance imaging (MRI) apparatus, the measurement coil thermoformed to follow a contour of a surface of a body of a subject of the MRI apparatus.

4. The method of claim 2, further comprising positioning the integrated circuitry of the wireless patient monitoring device between the thermoformed section of the substrate material and a non-thermoformed portion of the wireless patient monitoring device, and sealing the thermoformed section and the non-thermoformed portion to enclose the integrated circuitry;
wherein the non-thermoformed portion of the wireless patient monitoring device is one of:
a non-thermoformed section of the substrate material;
a non-thermoformed section of a second substrate material; and
a printed circuit board of the integrated circuitry.

5. The method of claim 4, wherein the thermoformed section and the non-thermoformed portion are sealed using one of an adhesive, thermoplastic staking, and ultrasonic welding.

6. The method of claim 1, wherein printing the electrical path on the substrate material using the conductive ink further comprises:
screen-printing a first layer of conductive ink on the substrate material;
drying the first layer at a first threshold temperature;
screen-printing a second layer of conductive ink on the substrate material on top of the first layer; and
drying the second layer at the first threshold temperature.

7. The method of claim 6, further comprising printing one or more additional dielectric layers on top of the conductive ink of the first and/or second layers of conductive ink.

8. The method of claim 1, wherein thermoforming the section of the substrate material including the electrical path using the mold further comprises:
heating the substrate material to a second threshold temperature;
stretching the substrate material over the mold, in a first thermoforming stage;
applying a vacuum suction through and around the mold at a first side of the substrate material, in a second thermoforming stage; and
applying a pressure at a second, opposite side of the substrate material, in a third thermoforming stage.

9. The method of claim 8, where the first thermoforming stage, the second thermoforming stage, and the third thermoforming stage are performed either concurrently or sequentially.

10. The method of claim 1, wherein the mold is selected from a plurality of molds based on the printed electrical path, the mold having differently shaped portions for areas of the printed electrical path and areas of the substrate material not including the printed electrical path, such that when the substrate material is stretched over the selected mold, the areas including the printed electrical path may be stretched less than the areas not including the printed electrical path.

11. The method of claim 1, wherein the substrate material is one of polyethylene terephthalate glycol (PETG), polystyrene or high impact polystyrene (HIPS), ethylene-vinyl acetate, and a polycarbonate (PC) polymer, and the conductive ink is a silver-based ink.

12. A wireless patient monitoring device, comprising:
an integrated circuitry including a sensing component and a wireless transmitter;
wherein the integrated circuitry is enclosed in a plastic film on which one or more printed electrical traces with a conductive ink are positioned, where the printed electrical traces comprise an antenna used by the wireless transmitter to transmit data acquired by the sensing component to a wireless receiver outside the wireless patient monitoring device.

13. The wireless patient monitoring device of claim 12, wherein the plastic film enclosing the integrated circuitry is thermoformed to create a hollow, domed shape in which the integrated circuitry is enclosed.

14. The wireless patient monitoring device of claim 12, wherein the sensing component is enclosed within a second thermoformed portion of the plastic film.

15. The wireless patient monitoring device of claim 14, wherein the wireless patient monitoring device is manufactured in a plurality of stages, including:
a first stage in which the one or more printed electrical traces are printed on a substrate material in the conductive ink;
a second stage in which a section of the substrate material including the one or more printed electrical traces comprising the antenna is heated and mechanically stretched using a mold;
a third stage in which a vacuum is applied through and around the mold at a first side of the substrate material; and
a fourth stage in which the integrated circuitry is coupled to, enclosed, and sealed by a thermoformed portion of the substrate material created by the second and third stages.

16. The wireless patient monitoring device of claim 15, wherein the third stage further comprises applying a pressure at a second, opposite side of the substrate material.

17. The wireless patient monitoring device of claim 15, in which the substrate material includes a first foldable portion that may be folded over the sensing component to enclose the sensing component, and a second foldable portion that may be folded over the integrated circuitry to enclose the integrated circuitry.

18. The wireless patient monitoring device of claim 15, wherein the mold is configured such that when the substrate material is stretched over the mold, some portions of the one or more printed electrical traces are stretched more than other portions of the one or more printed electrical traces.

19. A wireless radio-frequency (RF) coil unit of a magnetic resonance imaging (MRI) machine, comprising:
a substrate material on which one or more printed electrical traces of a conductive ink are positioned, wherein the one or more printed electrical traces comprise an imaging antenna that measures a realigning of protons within a portion of an anatomy of a subject of the MRI machine after gradient stimulation of the portion during an MR imaging scan, the substrate material thermoformed to follow a contour of a surface of the portion of the anatomy.

20. The wireless RF coil unit of claim 19, wherein the wireless RF coil unit is customized to the subject.
